(19) United States
(12) Patent Application Publication
(10) Pub. No.: US 2022/0287672 A1
(43) Pub. Date: Sep. 15, 2022

(54) RADIATION IMAGE CAPTURING SYSTEM, RADIATION IMAGE CAPTURING METHOD, MEDICAL IMAGE CAPTURING SYSTEM, AND STORAGE MEDIUM

(71) Applicant: CANON KABUSHIKI KAISHA, Tokyo (JP)

(72) Inventor: Tomohiko Matsuura, Tokyo (JP)

(21) Appl. No.: 17/826,854

(22) Filed: May 27, 2022

Related U.S. Application Data
(63) Continuation of application No. PCT/JP2020/043787, filed on Nov. 25, 2020.

(30) Foreign Application Priority Data
Dec. 3, 2019 (JP) 2019-218931

(51) Int. Cl. A61B 6/00; G06T 7/33; G06T 7/00

(52) U.S. Cl. CPC A61B 6/5264; A61B 6/463; A61B 6/5294; G06T 7/337; G06T 7/0012; G06T 2207/30004; G06T 2207/20212

(57) ABSTRACT

A radiation image capturing system includes a first acquisition unit configured to acquire a radiation image based on radiation irradiating a subject, a second acquisition unit configured to acquire a moving image composed of a plurality of optical images, by optically imaging the subject, a determination unit configured to determine whether alignment of the subject with respect to a radiation image capturing apparatus is completed, based on information about movement of the subject, and a display control unit configured to display a reference image indicating a state where the alignment of the subject is completed, among the plurality of optical images composing the moving image, together with the moving image, on a display unit, in a case where the determination unit determines that the alignment is completed.

### CROSS-REFERENCE TO RELATED APPLICATIONS

This application is a Continuation of International Patent Application No. PCT/JP2020/043787, filed Nov. 25, 2020, which claims the benefit of Japanese Patent Application No. 2019-218931, filed Dec. 3, 2019, both of which are hereby incorporated by reference herein in their entirety.

### BACKGROUND OF THE INVENTION

### Field of the Invention

The present invention relates to a radiation image capturing system, a radiation image capturing method, a medical image capturing system, and a storage medium.

### Background Art

In image capturing using a radiation image capturing system in the medical field, an inspector adjusts the position and the posture of a subject, moves from an imaging room to an operation room, and operates an exposure switch, thereby executing radiation image capturing. However, there is an issue that, if a change occurs in the position or posture of the subject while the inspector is moving, image recapturing is necessary depending on the degree of the change.

In recent years, to address the above-described issue, there has been a system having the following configuration.

PLT 1 discusses a technique in which an optical camera is attached to a radiation generating apparatus, and a misalignment amount is calculated based on an optical image immediately after the position and the posture of a subject are adjusted, and an optical image immediately before radiation irradiation. Subsequently, in a case where the calculated misalignment amount is determined to be out of tolerance, an inspector is notified of information indicating that there is misalignment.

### CITATION LIST

### Patent Literature

PTL 1: Japanese Patent Application Laid-Open No. 2011-024721

In the technique of PLT 1, however, there is an issue that a user operation is necessary to store the optical image immediately after the adjustment of the position and the posture of the subject, which is troublesome.

### SUMMARY OF THE INVENTION

In view of such an issue, the present invention is directed to reduction of the frequency of image recapturing without increasing the trouble of operation by an inspector.

A radiation image capturing system includes a first acquisition unit configured to acquire a radiation image based on radiation irradiating a subject, a second acquisition unit configured to acquire a moving image composed of a plurality of optical images, by optically imaging the subject, a determination unit configured to determine whether alignment of the subject with respect to a radiation image capturing apparatus is completed, based on information about movement of the subject, and a display control unit configured to display a reference image indicating a state where the alignment of the subject is completed, among the plurality of optical images composing the moving image, together with the moving image, on a display unit, in a case where the determination unit determines that the alignment is completed.

Further features of the present invention will become apparent from the following description of exemplary embodiments with reference to the attached drawings.

### BRIEF DESCRIPTION OF THE DRAWINGS

FIG. 1 is a diagram illustrating an example of a system configuration of a radiation image capturing system according to a first exemplary embodiment.

FIG. 2 is a diagram illustrating an example of a hardware configuration of a radiation image capturing control apparatus according to the first exemplary embodiment.

FIG. 3 is a diagram illustrating an example of a functional configuration of the radiation image capturing control apparatus according to the first exemplary embodiment.

FIG. 4 is a flowchart illustrating an example of a series of processing steps of the radiation image capturing control apparatus according to the first exemplary embodiment.

FIG. 5 is a diagram illustrating an example of a configuration of a guide video image in subject image capturing of the radiation image capturing control apparatus according to the first exemplary embodiment.

FIG. 6 is a diagram illustrating an example of a configuration of a radiation image capturing control apparatus according to a second exemplary embodiment.

FIG. 7 is a flowchart illustrating an example of a series of processing steps of the radiation image capturing control apparatus according to the second exemplary embodiment.

### DESCRIPTION OF THE EMBODIMENTS

Preferred exemplary embodiments of a radiation image capturing system according to the present invention will be described in detail below with reference to the attached drawings. However, components described in the exemplary embodiments are merely examples, and the technical scope of the radiation image capturing system according to the present invention is defined by the scope of claims, and not limited by the following specific exemplary embodiments. In addition, the present invention is not limited to the following exemplary embodiments according to the present invention, and various modifications (including organic combinations of some or all of the exemplary embodiments) can be made based on the gist of the present invention, and those are not excluded from the scope of the present invention. In other words, all of configurations obtained by combining some or all of the exemplary embodiments to be described below and modifications thereof are included in exemplary embodiments of the present invention.

Here, first, terms to be used in the present exemplary embodiments will be defined.

Positioning adjustment means that an inspector moves a subject to a position between a radiation generating apparatus and a radiation image capturing apparatus, and aligns the subject so that an imaging part of the subject is included in a region (an irradiation field) to be irradiated with radiation. The positioning adjustment also means that the inspector determines the posture of the subject so that an appropriate incidence angle of the radiation is achieved.

In addition, in the following, an image captured by the radiation image capturing apparatus will be described as “radiation image”, and an image optically captured by an optical camera or the like will be described as “optical image”. In the following exemplary embodiments, a plurality of optical images in series obtained in time order will be described as “moving image”. Each of the plurality of optical images composing the moving image will be described as “frame image”.

### First Exemplary Embodiment

A radiation image capturing system according to the present exemplary embodiment captures a moving image of a subject, using an optical camera attached to a radiation generating apparatus. Further, the radiation image capturing system automatically determines whether the adjustment of the position and the posture of the subject is completed, and acquires a frame image of the subject at the time of the completion of the adjustment. Subsequently, the radiation image capturing system displays the captured moving image and the frame image of the subject at the time of the completion of the adjustment in a superimposed manner on a display unit. An inspector can thereby recognize without operational time and effort whether the position and the posture of the subject are not shifted from those at the time of the completion of the adjustment, before radiation irradiation, while viewing the two optical images superimposed on each other, so that an imaging failure attributable to a shift of the position or posture of the subject can be prevented. In other words, the frequency of image recapturing can be reduced.

In the present exemplary embodiment, the radiation image capturing system is described as an example, but a medical image capturing system using a magnetic resonance imaging (MRI) apparatus, an ultrasound imaging apparatus, a photoacoustic tomography apparatus, or the like may be used. In other words, the present invention is applicable to any type of system if the system uses a medical image capturing apparatus in which an imaging failure attributable to misalignment of a subject can occur.

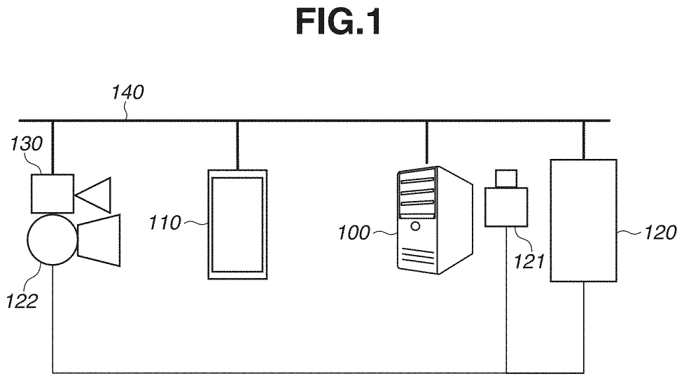

A system configuration of the present exemplary embodiment will be described with reference to FIG. 1 to FIG. 3.

FIG. 1 illustrates a configuration example of the entire information processing system of the present exemplary embodiment. This system is composed of a radiation image capturing control apparatus 100, a radiation image capturing apparatus 110, a radiation generating apparatus 120, and an optical image acquisition apparatus 130 via a network 140. The network 140 may be a wired network or a wireless network.

The radiation image capturing control apparatus 100 is an apparatus configured of an information processing apparatus such as a computer, which communicates with the radiation image capturing apparatus 110, and controls the radiation image capturing. The radiation image capturing control apparatus 100 communicates with the radiation generating apparatus 120, and acquires information at the time of radiation irradiation, from the radiation generating apparatus 120. The radiation image capturing control apparatus 100 also communicates with the optical image acquisition apparatus 130, controls the optical image acquisition apparatus 130, and acquires an optical image captured by the optical image acquisition apparatus 130.

The radiation image capturing apparatus 110 is an apparatus that shifts to an image-capturing enabled state based on an instruction from the radiation image capturing control apparatus 100, performs radiation image capturing while synchronizing with the radiation generating apparatus 120, and generates an image based on radiation irradiation by the radiation generating apparatus 120. The number of the radiation image capturing apparatuses 110 is not limited to one, and a configuration in which a plurality of radiation image capturing apparatuses is used may be employed.

The radiation generating apparatus 120 is an apparatus that detects a radiation irradiation instruction given by an exposure switch 121, and generates radiation from an X-ray tube 122, based on irradiation information set by a user input device (not illustrated) that accepts user operations, such as an operation panel.

The optical image acquisition apparatus 130 is an apparatus that performs image capturing based on an instruction from the radiation image capturing control apparatus 100, and acquires an optical image of a subject in real time. In the present exemplary embodiment, the optical camera is used as the optical image acquisition apparatus 130, but the configuration is not limited if an optical image can be acquired. In the present exemplary embodiment, the optical image acquisition apparatus 130 is attached to the X-ray tube 122, and performs image capturing in the radiation generation direction of the X-ray tube 122.

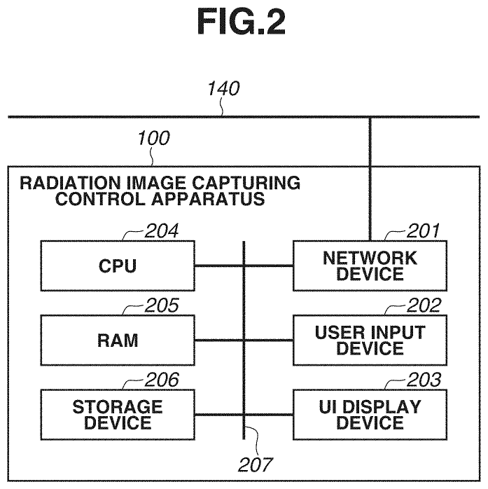

FIG. 2 is a hardware configuration example of the radiation image capturing control apparatus 100 of the radiation image capturing system of the present exemplary embodiment.

The radiation image capturing control apparatus 100 has a network device 201 that connects with the network 140, and a user input device 202 that accepts user operations, such as a keyboard.

The radiation image capturing control apparatus 100 further has a user interface (UI) display device 203 that displays an operation screen and a radiation image, such as a liquid crystal display, and a central processing unit (CPU) 204 that controls the entire apparatus.

The radiation image capturing control apparatus 100 further has a random access memory (RAM) 205 that provides a work space of the CPU 204, and a storage device 206 that stores various control programs, radiation images received from the radiation image capturing apparatus 110, image information received from the optical image acquisition apparatus 130, and the like.

Here, the devices included in the radiation image capturing control apparatus 100 are each connected by a main bus 207, and can transmit and receive data to and from each other.

The user input device 202 and the UI display device 203 are described as separate devices, but these devices may be integrated to form an operation unit.

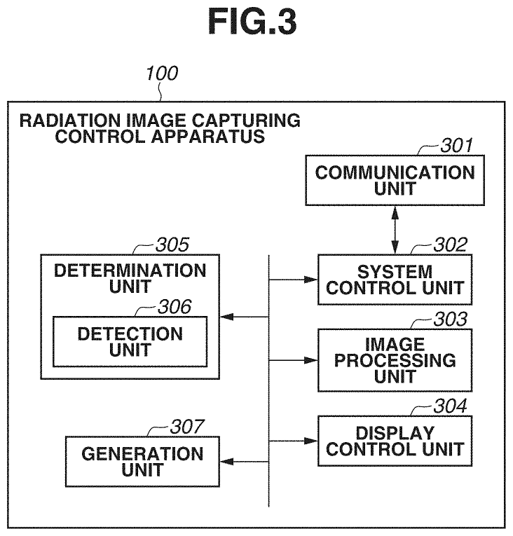

FIG. 3 is a functional configuration example of the radiation image capturing control apparatus 100 of the radiation image capturing system of the present exemplary embodiment.

The CPU 204 on the radiation image capturing control apparatus 100 reads out a control program stored in the storage device 206 into the RAM 205, and executes the control program, thereby implementing each functional unit illustrated in FIG. 3.

The radiation image capturing control apparatus 100 has a communication unit 301, a system control unit 302, an image processing unit 303, a display control unit 304, a determination unit 305, and a generation unit 307.

The communication unit 301 is software that performs communication by controlling the network device 201.

The system control unit 302 controls the optical image acquisition apparatus 130, acquires irradiation information of the radiation generating apparatus 120 and imaging information of the radiation image capturing apparatus 110, and manages the state of each of the apparatuses, via the communication unit 301.

Further, the system control unit 302 acquires each of a captured radiation image from the radiation image capturing apparatus 110, and an optical image from the optical image acquisition apparatus 130, via the communication unit 301.

Furthermore, the system control unit 302 is a program that implements a basic function of the radiation image capturing control apparatus 100, and controls the operation of each unit.

The image processing unit 303 processes the captured radiation image acquired via the system control unit 302, and generates an image to be used on the radiation image capturing control apparatus 100.

The display control unit 304 displays the image generated by the image processing unit 303, via the UI display device 203.

Further, the display control unit 304 displays a guide video image generated by the generation unit 307, via the UI display device 203.

Furthermore, the display control unit 304 performs reflection of processing in an image in accordance with an instruction from the system control unit 302 based on an operation from the user input device 202, processing of switching the screen of the UI display device 203, and the like.

The determination unit 305 determines the completion of the positioning adjustment, based on an optical image obtained from the optical image acquisition apparatus 130. In the present exemplary embodiment, the determination unit 305 has a detection unit 306 that detects movement information of a subject in a moving image, and determines whether the positioning adjustment is completed, based on the information about the movement of the subject detected by the detection unit 306. For example, the determination unit 305 determines that the positioning adjustment is completed, in a case where a state where there is no movement of the subject is detected.

Here, the state where there is no movement indicates, for example, such a state that a state where a movement amount of the subject is below a predetermined threshold continues for a time longer than a certain period of time. Specifically, for example, in a case where the threshold of the movement amount is 5 cm and the time for the continuation is 5 seconds, a state where the movement amount of the subject is below 5 cm continues for a time longer than 5 seconds is the state where there is no movement. The above-described method of setting the threshold is an example, and, for example, such a state that a state where the movement amount is less than 3 cm continues for a time longer than 3 seconds may be the state where there is no movement. The example in which the threshold is not included is described, but whether to include the threshold in setting of the threshold can be appropriately designed. In addition, a threshold varying from part to part of the subject may be set.

For the specific processing method of the detection unit 306, i.e., the method of detecting the movement information from the moving image, for example, a method based on a differential value in terms of signal between the frame images composing the moving image can be used. The detection method is not limited to the one described above, and may be any method if the movement information can be detected from the moving image.

A region from which the detection unit 306 detects the movement information may be the entire region within the moving image, or may be a partial region thereof. For example, in a case where a target part for performing radiation image capturing is an arm, a region where the arm appears may be the region for detecting the movement information, and only the movement information about the arm may be detected. In this case, the determination unit 305 determines whether the positioning adjustment is completed, based on the information about the movement of the subject in a partial region detected by the detection unit 306.

The generation unit 307 generates the guide image, based on the optical image obtained from the optical image acquisition apparatus 130, and the presence/absence of a notification for notifying the completion of the positioning adjustment received from the determination unit 305. In the present exemplary embodiment, the generation unit 307 generates the moving image itself as the guide image, until the generation unit 307 receives the notification for notifying the completion of the positioning adjustment. Further, upon receiving the notification for notifying the completion of the positioning adjustment, the generation unit 307 superimposes the frame image at the time of the completion of the positioning adjustment (hereinafter referred to as the reference image) on the moving image, and generates the guide image. The reference image is not limited to the frame image at the time of the completion of the positioning adjustment, and may be selectively acquired from a plurality of frame images including a predetermined number of frame images captured temporally near the frame image at the time of the completion. Specifically, for example, in a case where the predetermined number is 2, the reference image is acquired from five frame images in total, including two frames before and after the frame image at the time of the completion of the positioning adjustment. This makes it possible to acquire a desired reference image, even if the timing when the positioning is completed and the timing of determining the positioning completion by the determination unit 305 do not coincide.

Afterward, the generation unit 307 instructs the display control unit 304 to display the generated guide image.

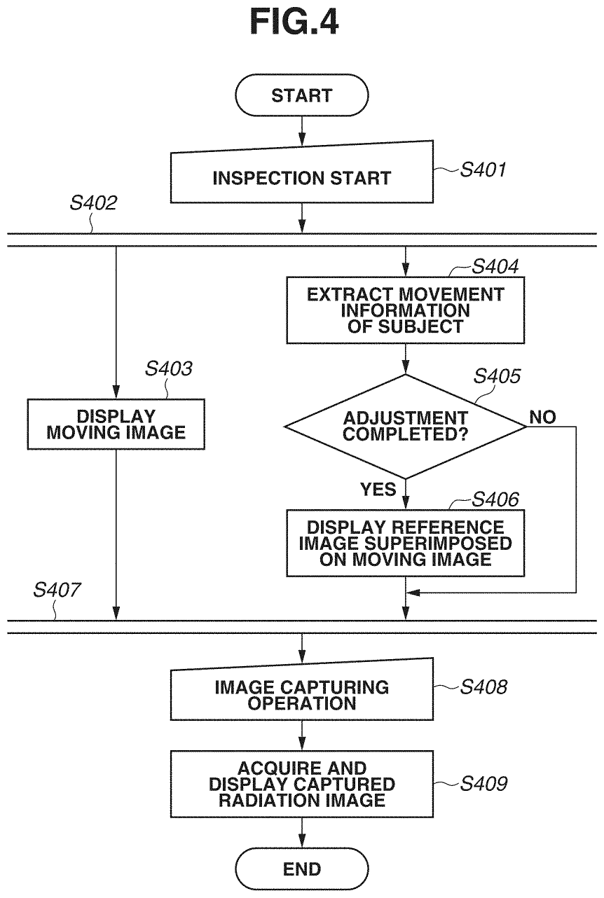

FIG. 4 is a flowchart illustrating an example of a series of display processing steps in subject image capturing of the radiation image capturing control apparatus 100.

In step S401, the system control unit 302 brings the radiation image capturing control apparatus 100 into an inspection start state for controlling image capturing, based on a user operation. Specifically, the system control unit 302 transmits an instruction to prepare for image capturing to the radiation image capturing apparatus 110 via the communication unit 301, based on the image capturing condition of a subject for which an inspection is ordered by a user operation. The radiation image capturing apparatus 110 transmits a preparation completion notification to the radiation image capturing control apparatus 100 in return, upon completing the image capturing preparation of itself. Upon receipt of the preparation completion notification, the system control unit 302 brings the radiation image capturing control apparatus 100 into an image-capturing enabled state to accept step S408 to be described below. The system control unit 302 also transmits an instruction to start image capturing to the optical image acquisition apparatus 130, via the communication unit 301. Upon receiving the image capturing start instruction, the optical image acquisition apparatus 130 sequentially transmits a moving image acquired by itself to the radiation image capturing control apparatus 100, in return.

Between step S402 and step S407, the system control unit 302 executes sequential parallel processing. Specifically, step S403, step S404 to step S406, and control processing except for these steps, and acceptance of user control are executed. The system control unit 302 executes the processing between the above-described steps, until step S408 is executed, or the inspection is cancelled by a user operation (not illustrated).

In step S403, the system control unit 302 displays the moving image acquired from the optical image acquisition apparatus 130 via the communication unit 301, on the UI display device 203, via the display control unit 304.

In step S404, the detection unit 306 detects the movement information of the subject within the moving image, based on the moving image acquired via the system control unit 302.

In step S405, the determination unit 305 determines whether the positioning adjustment is completed, based on the movement information of the subject detected from the moving image by the detection unit 306.

If the positioning adjustment is determined to be completed in step S405, then in step S406, the display control unit 304 displays the reference image generated by the generation unit 307 and the moving image displayed on the UI display device 203, in a superimposed manner.

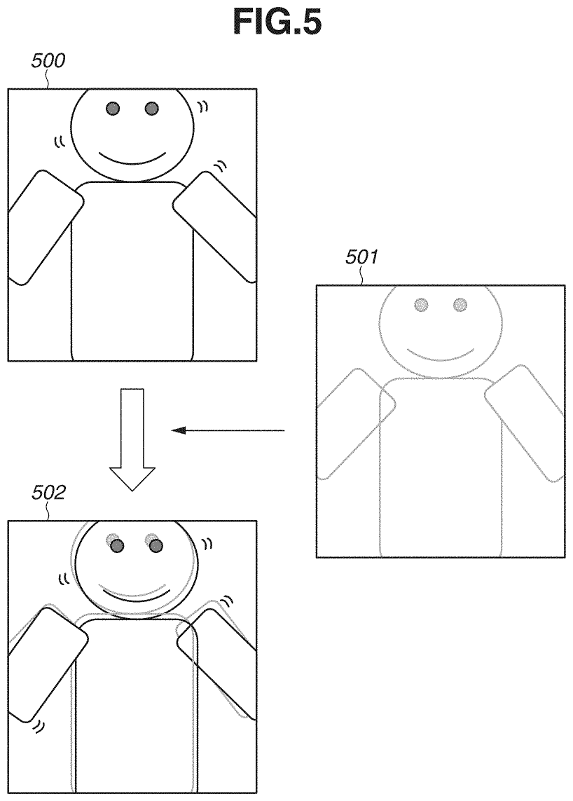

Here, a configuration about the display of the moving image, the reference image, and the guide image displayed on the UI display device 203 in step S403 to step S406 will be described with reference to FIG. 5.

A moving image 500 is a plurality of optical images obtained in time sequence, which is the moving image acquired from the optical image acquisition apparatus 130 and displayed on the UI display device 203 in step S403. In the actual moving image, an object within the image capturing range of the optical image acquisition apparatus 130 appears due to the presence of the radiation image capturing apparatus 110 behind the subject, or the like, but a diagram illustrating only the physical information of the subject in the moving image 500 is used for the purpose of description. For the subsequent optical images as well, a diagram illustrating only the physical information of the subject will be used unless otherwise specified.

A reference image 501 is an optical image generated from one frame of the optical image at the time of the completion of the positioning adjustment or several frames near the one frame, as described above.

A guide image 502 is the guide image generated by superimposing the reference image 501 and the moving image 500 on each other in step S406, and displayed on the UI display device 203.

The example in which the display control unit 304 displays the reference image 501 and the moving image 500 in a superimposed manner is described above, but, for example, the reference image 501 and the moving image 500 may be displayed side by side so that the inspector can recognize the misalignment of the subject. In other words, the display control unit 304 corresponds to an example of a display control unit that displays a reference image indicating a state where the alignment of a subject is completed, together with a moving image, on a display unit.

The description will continue referring back to the flowchart in FIG. 4.

In step S408, the user presses the exposure switch 121 of the radiation generating apparatus 120, so that the image capturing begins. When the image capturing begins, the radiation generating apparatus 120 generates radiation from the X-ray tube 122, and the radiation that has passed through the subject is notified to the radiation image capturing apparatus 110, and the radiation image capturing apparatus generates a radiation image. The radiation generating apparatus 120 may be configured not to generate the radiation in a case where the subject is shifted from the position appearing in the reference image by a certain amount or more in step S408. This can reduce the possibility of the execution of image capturing in a state where there is misalignment of the subject.

In step S409, the system control unit 302 transfers the radiation image generated in step S408 to the radiation image capturing control apparatus 100, generates a radiation image for diagnoses, using the image processing unit 303, and displays the generated radiation image on the UI display device 203, using the display control unit 304.

The processing of the radiation image capturing system according to the present exemplary embodiment is thus performed.

According to the foregoing, in the first exemplary embodiment, the radiation image capturing control apparatus 100 displays the moving image acquired from the optical image acquisition apparatus 130 at the time of the start of the positioning adjustment, on the UI display device 203. Subsequently, the radiation image capturing control apparatus 100 can acquire the frame image as the reference image without receiving a special user operation at the time of the completion of the positioning adjustment, and can display the guide image obtained by superimposition on the moving image on the UI display device 203. In other words, the user who has moved from an imaging room to an operation room can carry out the radiation image capturing at appropriate timing, while confirming the optical image at the time of the positioning completion and the current moving image that are displayed in a superimposed manner.

### Second Exemplary Embodiment

Next, a second exemplary embodiment of the present invention will be described.

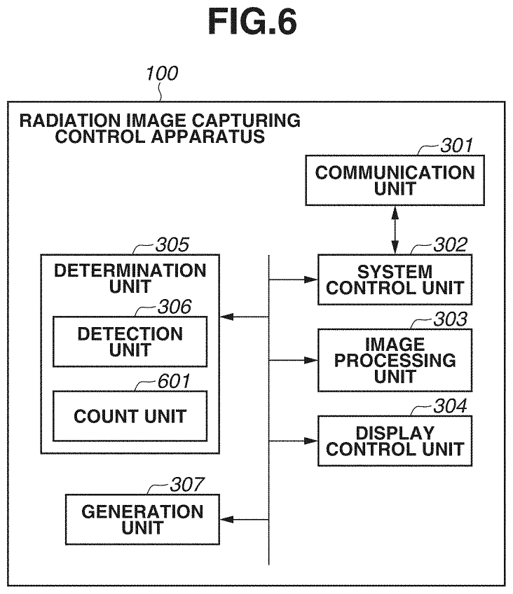

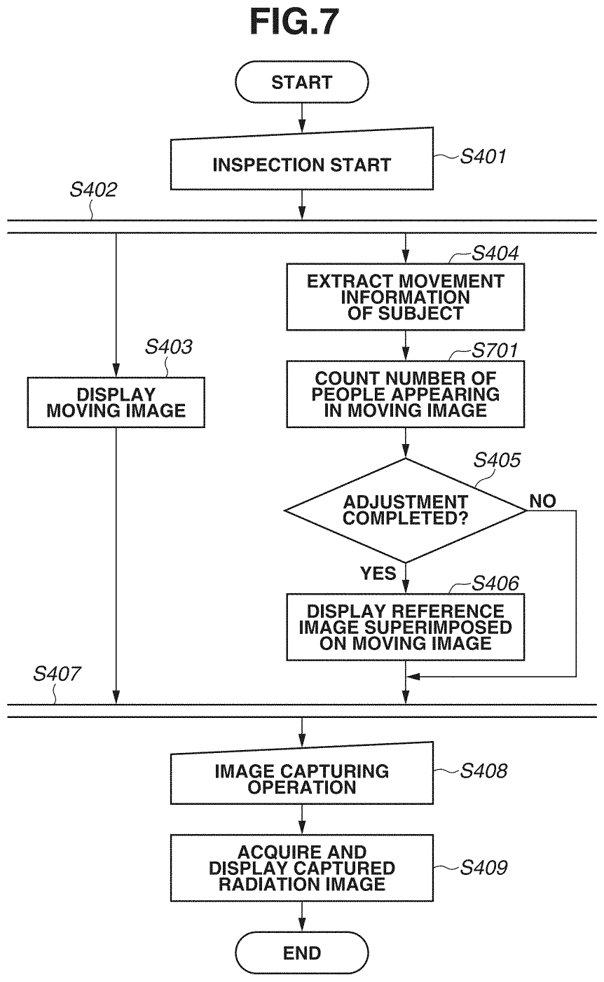

In a configuration of the second exemplary embodiment, processing of detecting the number of subjects appearing in an optical image in the determination of the completion of the positioning adjustment by the radiation image capturing control apparatus 100 is added. Only a part different from the first exemplary embodiment will be described below with reference to FIG. 6 and FIG. 7.

FIG. 6 is a configuration example of the radiation image capturing control apparatus 100 of the radiation image capturing system of the present exemplary embodiment. The radiation image capturing control apparatus 100 additionally has a count unit 601.

The count unit 601 counts the number of people appearing in an optical image, using an optical image obtained from the optical image acquisition apparatus 130. In the present exemplary embodiment, the count unit 601 counts the number of people, using an inference device that holds a feature amount about the shape of a human body image obtained in pretraining. A specific method to be used for machine learning in the present exemplary embodiment is not limited, and, as an architecture of convolutional neural network, for example, a region-based CNN (R-CNN), or a method obtained by combining a plurality of other methods, may be used. In addition, the specific method is not limited if the number of people appearing in an optical image can be counted, and the specific method may be any of many existing known techniques or a combination of some of those techniques, without being limited to the method to be used for machine learning.

FIG. 7 is a flowchart of display processing at the time of subject image capturing of the radiation image capturing control apparatus 100 of the present exemplary embodiment.

In step S701, the count unit 601 counts the number of people appearing in a moving image acquired via the system control unit 302.

In step S405, the determination unit 305 determines whether the positioning adjustment is completed, based on the movement information of the subject detected from the moving image by the detection unit 306 and the number of people counted by the count unit 601. The specific determination method is not particularly limited, but in the present exemplary embodiment, the determination unit 305 determines that the positioning adjustment is completed, in a case where the magnitude of a movement of the subject is less than a predetermined threshold. Alternatively, the determination unit 305 determines that the positioning adjustment is completed, in a case where the number of people appearing in the optical image is one. Alternatively, the determination unit 305 determines that the positioning adjustment is completed, in a case where the magnitude of a movement of the subject is less than a predetermined threshold and the number of people appearing in the optical image is one.

The determination unit 305 may determine whether the positioning adjustment is completed, based on only the number of people counted by the count unit 601. In this case, the inspector can support the subject to maintain the posture immediately before the determination of the positioning completion, even if it is difficult for the subject to stay still for a certain period of time because of old age or an injury, and therefore, it is possible to perform the determination irrespective of the state or physical condition of the subject. As a result, the user who has moved from the imaging room to the operation room can carry out the radiation image capturing at appropriate timing, while confirming the optical image at the time of the positioning completion and the current moving image that are displayed in a superimposed manner.

### Third Exemplary Embodiment

Next, a third exemplary embodiment of the present invention will be described.

In the present exemplary embodiment, the guide image is presented not only to the inspector but also to the subject so that the guide image is utilized to let the subject him/herself to replicate the position and the posture of the body at the time of the positioning adjustment completion.

Specifically, there is provided a configuration (not illustrated) in which another UI display device 203 is added for the presentation to the subject, and the display control unit 304 additionally has a control function for displaying a horizontally flipped optical image on this UI display device 203.

In the third exemplary embodiment, the above-described configuration enables the subject him/herself to confirm the optical image, so that the replicability of the position and the posture of the body at the time of the positioning adjustment completion can be improved. As a result, the time and effort taken before obtaining appropriate timing for radiation image capturing can be reduced, and moreover, the burden on the inspector and the subject can be alleviated.

The present invention is not limited to the above embodiments and various changes and modifications can be made within the spirit and scope of the present invention. Therefore, to apprise the public of the scope of the present invention, the following claims are made.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present invention, it is possible to reduce the frequency of image recapturing without increasing the trouble of operation by an inspector.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation image capturing system comprising:
a first acquisition unit configured to acquire a radiation image based on radiation irradiating a subject;
a second acquisition unit configured to acquire a moving image composed of a plurality of optical images, by optically imaging the subject;
a determination unit configured to determine whether alignment of the subject with respect to a radiation image capturing apparatus is completed, based on information about movement of the subject; and
a display control unit configured to display a reference image indicating a state where the alignment of the subject is completed, among the plurality of optical images composing the moving image, together with the moving image, on a display unit, in a case where the determination unit determines that the alignment is completed.

2. The radiation image capturing system according to claim 1, further comprising a detection unit configured to detect movement of the subject,
wherein the determination unit determines whether the alignment is completed, based on information about the movement of the subject detected by the detection unit.

3. The radiation image capturing system according to claim 2, wherein the determination unit determines that the alignment is completed, in a case where a state where there is no movement of the subject is detected by the detection unit.

4. The radiation image capturing system according to claim 3, wherein the determination unit determines a state where a movement amount of the subject per predetermined time is less than a threshold, as the state where there is no movement.

5. The radiation image capturing system according to claim 2,
wherein the detection unit detects movement of the subject in a partial region of the moving image, and
wherein the determination unit determines whether the alignment is completed, based on information about the movement of the subject in the partial region detected by the detection unit.

6. The radiation image capturing system according to claim 1, wherein the display control unit displays the reference image and the moving image in a superimposed manner on the display unit.

7. The radiation image capturing system according to claim 1, wherein the display control unit displays the reference image obtained from a frame image at a time when the alignment of the subject is completed and a predetermined number of frame images captured temporally near the frame image, together with the moving image, on the display unit.

8. The radiation image capturing system according to claim 1,
wherein the determination unit includes a unit of counting a number of people appearing in the moving image, and
wherein the display control unit displays the reference image together with the moving image on the display unit, in a case where the determination unit determines that the alignment of the subject is completed, and the number of people appearing in the moving image is one.

9. The radiation image capturing system according to claim 1, wherein the display control unit can display the moving image and the reference image displayed on the display unit, in a horizontally flipped state.

10. The radiation image capturing system according to claim 1, wherein the second acquisition unit acquires the moving image by imaging the subject in real time.

11. The radiation image capturing system according to claim 1, wherein the display control unit selects the reference image indicating the state where the alignment of the subject is completed from the plurality of optical images composing the moving image, and displays the selected reference image together with the moving image on the display unit.

12. A radiation image capturing system comprising:
a first acquisition unit configured to acquire a radiation image based on radiation irradiating a subject;
a second acquisition unit configured to acquire a moving image composed of a plurality of optical images, by optically imaging the subject;
a determination unit configured to determine whether alignment of the subject with respect to a radiation image capturing apparatus is completed, based on information about a number of people appearing in the moving image of the subject; and
a display control unit configured to display a reference image indicating a state where the alignment of the subject is completed, among the plurality of optical images composing the moving image, together with the moving image, on a display unit, in a case where the determination unit determines that the alignment is completed.

13. A medial image capturing system comprising:
an acquisition unit configured to acquire a moving image composed of a plurality of optical images by optically imaging a subject;
a determination unit configured to determine whether alignment of the subject with respect to a medial image capturing apparatus is completed, based on information about movement of the subject; and
a display control unit configured to display a reference image indicating a state where the alignment of the subject is completed, among the plurality of optical images composing the moving image, together with the moving image, on a display unit, in a case where the determination unit determines that the alignment is completed.

14. A radiation image capturing method comprising:
Acquiring, in first acquisition, a radiation image based on radiation irradiating a subject;
acquiring, in second acquisition, a moving image composed of a plurality of optical images, by optically imaging the subject;
automatically determining, using a determination unit, whether alignment of the subject is completed, based on information about movement of the subject; and
controlling display to display a reference image indicating a state where the alignment of the subject is completed, among the plurality of optical images composing the moving image, together with the moving image, on a display unit, in a case where the alignment is determined to be completed.

15. A storage medium configured to store a program for causing a computer to execute the radiation image capturing method according to claim 14.
